(19) United States
(12) Patent Application Publication
(10) Pub. No.: US 2026/0354998 A1
(43) Pub. Date: Nov. 12, 2026

(54) KNEE BRACE AND KNEE BRACE STABILIZER ASSEMBLY

(71) Applicants: Tonna Grigsby, Sullivan, IN (US); Emily Lawrence, Terre Haute, IN (US); Shirish Sachdeva, Terre Haute, IN (US)

(72) Inventors: Tonna Grigsby, Sullivan, IN (US); Emily Lawrence, Terre Haute, IN (US); Shirish Sachdeva, Terre Haute, IN (US)

(21) Appl. No.: 19/648,118

(22) Filed: Apr. 15, 2026

Related U.S. Application Data
(60) Provisional application No. 63/800,562, filed on May 6, 2025.

(51) Int. Cl. A61F 5/01

(52) U.S. Cl. CPC A61F 5/0125; A61F 2005/0167

(57) ABSTRACT

A knee brace and knee brace stabilizer assembly, comprising a knee brace including a knee brace mechanism configured to restrain movement of a human knee and a knee strap coupled to the knee brace mechanism, a knee brace stabilizer including a waist band configured to be supported by a human waist, the waist band including a belt and a plurality of loops supported by the belt, and a connector strap that couples to at least one of the plurality of loops and couples to the knee strap to restrain movement of the knee brace relative to the waist band.

### RELATED APPLICATION

The present application claims the benefit of U.S. Provisional Patent Application Ser. No. 63/800,562, titled Knee Brace and Knee Brace Stabilizer Assembly, to Tonna Grigsby et al., filed on May 6, 2025, the entire disclosure of which is expressly incorporated by reference herein.

### TECHNICAL FIELD OF INVENTION

The present invention relates to a knee brace and knee brace stabilizer assembly.

### BACKGROUND AND SUMMARY OF THE DISCLOSURE

This section introduces aspects that may help facilitate a better understanding of the disclosure. Accordingly, these statements are to be read in this light and are not to be understood as admissions about what is or is not prior art.

Knee braces are commonly used to support and stabilize the knee joint, especially during post-injury rehabilitation. However, these braces can sometimes slide around and fail to prevent unwanted movements.

According to the present disclosure, a knee brace and knee brace stabilizer assembly is provided. The assembly comprises a knee brace including a knee brace mechanism configured to restrain movement of a human knee and a knee strap coupled to the knee brace mechanism. The assembly further comprises a knee brace stabilizer including a waist band configured to be supported by a human waist. The waist band includes a belt and a plurality of loops supported by the belt. The assembly further comprises a connector strap that couples to at least one of the plurality of loops and couples to the knee strap to restrain movement of the knee brace relative to the waist band.

According to another aspect of the present disclosure, a knee brace and knee brace stabilizer assembly is provided. The assembly comprises a knee brace including

a knee brace mechanism configured to restrain movement of a human knee and

a knee strap coupled to the knee brace mechanism. The assembly further comprises a knee brace stabilizer including a waist band configured to be supported by a human waist and a connector strap that couples to the waist band to restrain movement of the knee brace relative to the waist band. The connector strap has an adjustable length to permit adjustment of the distance between the waist band and the knee strap.

According to another aspect of the present disclosure, a knee brace and knee brace stabilizer assembly is provided. The assembly comprises a knee brace including

a knee brace mechanism configured to restrain movement of a human knee and

a knee strap coupled to the knee brace mechanism. The assembly further comprises a knee brace stabilizer including a waist band configured to be supported by a human waist and a connector strap that couples to waist band to restrain movement of the knee brace relative to the waist band. The connector strap has an upper portion coupled to the waist band, a lower portion coupled to the knee strap, and a connector connecting the upper portion to the lower portion.

### BRIEF DESCRIPTION OF THE DRAWINGS

The previously described aspects of this disclosure will grow to be appreciated at a greater level once references to the following accompanying illustrations are expounded upon.

FIG. 1 is an enlarged side view of a knee brace stabilizer assembly and a portion of a knee brace on a right knee of a person showing the knee brace stabilizer assembly including a waist band and two connector straps extending down from the waist band.

FIG. 2 is a rear view of the person using stabilizing bars with the knee brace and knee brace stabilizer assembly of FIG. 1 provided on the right knee of the person.

FIG. 3 is a front view of the main band of the waist band.

FIG. 4 is a back view of the main band of the waist band of FIG. 3.

FIG. 5 is a front view of an upper portion of one of the connector straps with a male connector.

FIG. 6 is a back view of the upper portion of the connector strap of FIG. 5.

FIG. 7 is a side view of the upper portion of the connector strap of FIG. 5.

FIG. 8 is a front view of a lower portion of one of the connector straps showing the lower portion of the connector strap having a female connector and hook and loop fasteners to allow adjustment of the length of the lower portion of the connector strap.

FIG. 9 is a front view of the lower portion of the connector strap shown in FIG. 8.

FIG. 10 is a back view of the lower portion of the connector strap of FIG. 8.

FIG. 11 is a front view of an extension band of the waist band.

FIG. 12 is a back view of the extension band of the waist band of FIG. 11.

FIG. 13 is a front perspective of an alternative embodiment main band with a sensor positioned for location in a pocket of the main band.

FIG. 14 is a back perspective view of the main band of FIG. 13.

The embodiments disclosed below are not intended to be exhaustive or limit the disclosure to the precise form disclosed in the following detailed description. Instead, the embodiments are chosen and described so that others skilled in the art may utilize their teachings. Unless otherwise indicated, the components shown in the figures are shown proportional to each other. It will be understood that no limitation of the scope of the disclosure is thereby intended. The disclosure includes any alterations and further modifications in the illustrative devices and described methods and further applications of the principles of the disclosure, which would normally occur to one skilled in the art to which the disclosure relates.

To promote an understanding of the principles of the present disclosure, reference will now be made to the embodiments illustrated in the drawings, and specific language will be used to describe the same. It will nevertheless be understood that no limitation of the scope of this disclosure is thereby intended.

### DETAILED DESCRIPTION

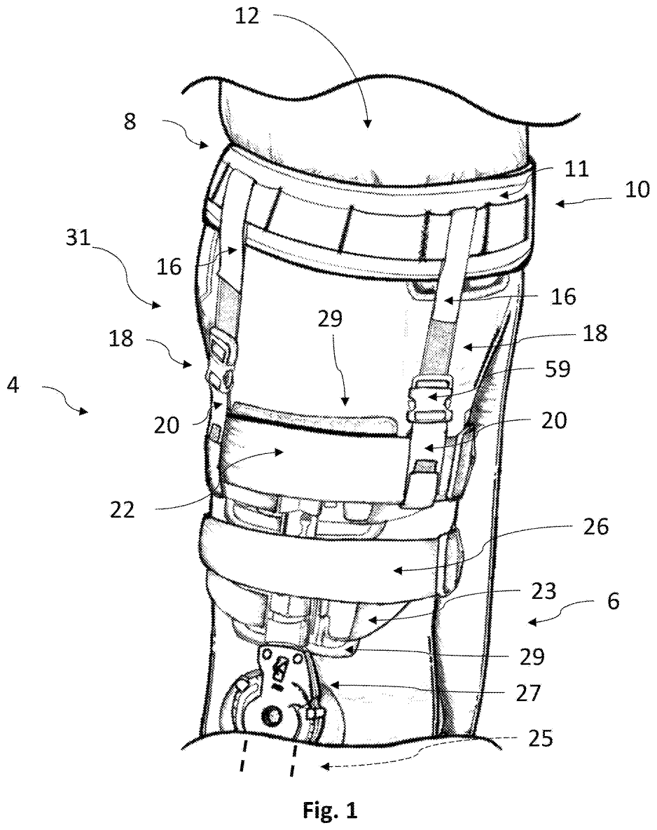

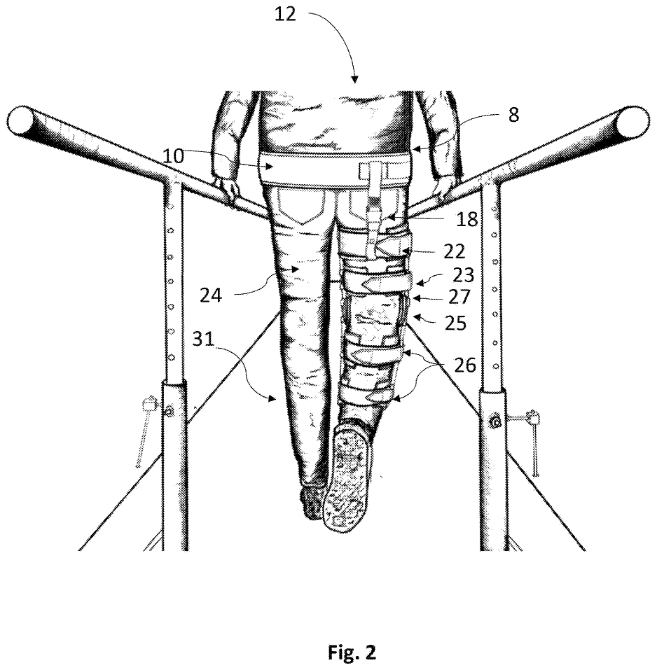

As shown in FIGS. 1 and 2, a knee brace stabilizer 4 is provided to support a knee brace mechanism 6 on a user 12. Knee brace stabilizer 4 includes a waist band 10 that is strapped around a waist 8 of user 12 and a plurality of connector straps 18 extending down from waist band 10. Waist band 10 has a main band 11 that has a plurality of loops 14 that couple to upper portions 16 of connector straps 18. Upper portions 16 interlock with a lower portion 20 of connector strap 18. Lower portions 20 couple to a knee strap 22, which wrap around and/or connects to knee brace mechanism 6.

In one embodiment, knee brace stabilizer 4 is secured to a single leg 24 of user 12. In such a use, at least two connector straps 18 are spaced apart from each other by coupling to different loops 14 of waist band 10 and coupling to different regions of knee strap 22 on single leg 24 of user 12 so that connector straps 18 are positioned on only one side (right or left) of user 12. For example, as shown in FIG. 2, knee brace stabilizer 4 is positioned on the right side/leg 24 of user 12 and not positioned on the left side/leg 24 of user 12. However, knee brace stabilizer 4 may be positioned on the left side of user 12 and not positioned on the right side of user 12, or knee brace stabilizers 4 may be provided on both the right and left legs 24 of user 12 as needed. Knee brace stabilizer 4 preferably extends no lower than knee brace mechanism 6.

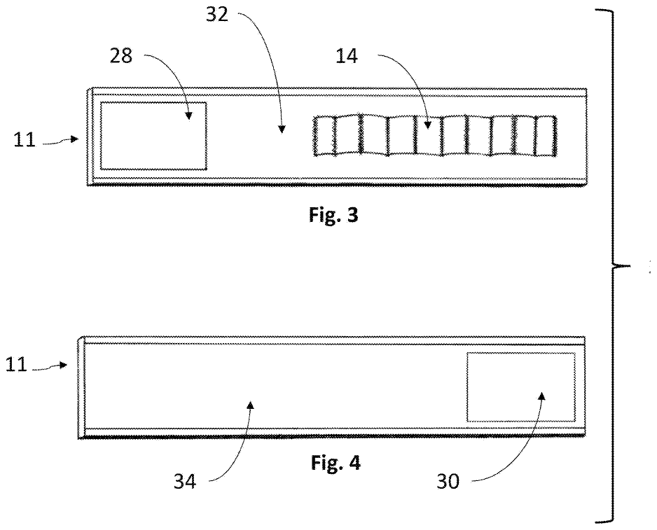

As mentioned above, main band 11 has a plurality of loops 14 that face away from user 12, as shown in FIGS. 3 and 4. Main band 11 also has a first patch/area 28 of hook and loop fasteners, and a second patch/area 30 of hook and loop fasteners. Additionally, main band 11 has a first side 32 and a second side 34. In one design, first patch 28 has hook fasteners on first side 32, and second patch 30 has loop fasteners on the second side 34. Main band 11 goes around waist 8 of user 12 and is secured by fastening first patch 28 with second patch 30. Typically, waistband 10 and knee brace 6 go over clothing 31 of user 12 and is not integrated into clothing 31. Plurality of loops 14 are also evenly spaced apart and are wide enough to permit upper portion 16 to extend through loops 14.

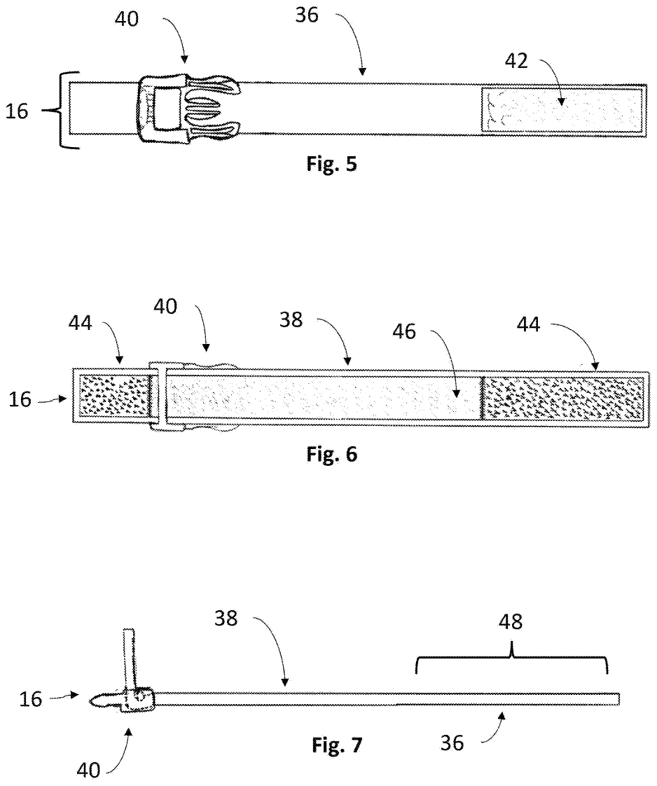

Upper portion 16 of connector strap 18 includes a first-upper portion side 36, a second upper-portion side 38, and a male connector 40, as shown in FIGS. 5, 6, and 7. Additionally, upper portion 16 has a first upper-portion patch/area 42 of hook and loop fasteners, a plurality of upper-portion patches 44 of hook and loop fasteners, and a second upper-portion patch/area 46 of hook and loop fasteners. In one design, first upper-portion patch 42 has loop fasteners on first upper-portion side 36, plurality of upper-portion patches 44 has hook fasteners on second upper-portion side 38, and second upper-portion patch 46 has loop fasteners on second upper-portion side 38. As such, first upper-portion patch 42 can fasten to plurality of upper-portion patches 44, and second upper-portion patch 46 can fasten to plurality of upper-portion patches 44. These fastening interfaces assist the upper portion 16 from sliding up or down. A length 48 of upper portion 16 can go through plurality of loops 14 such that first connector 40 extends between waist band 10 and a female connector 50 of lower portion 20 of connector strap 18.

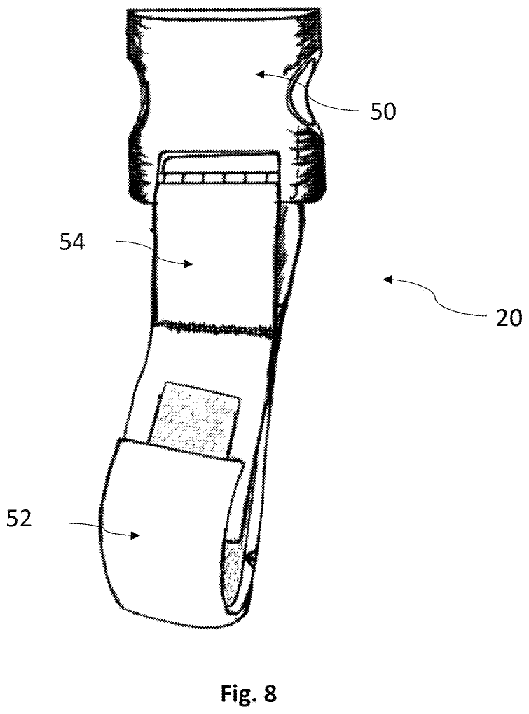

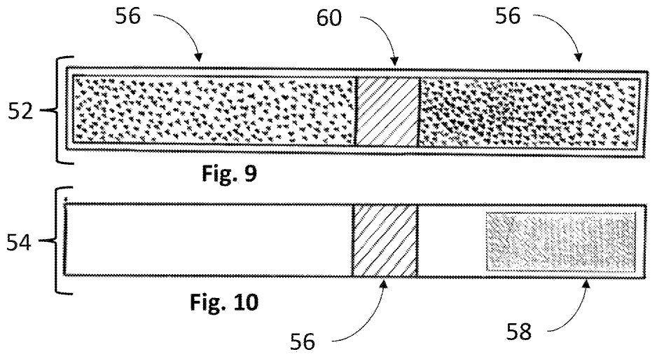

As shown in FIGS. 8, 9 and 10, lower portion 20 of connector strap 18 includes female connector 50, a first lower strap 52, and a second lower strap 54. First lower strap 52 has a plurality of lower portion patches/areas 56 of hook fasteners, and second lower strap 54 has a lower portion patch/area 58 of loop fasteners. Female connector 50 connects to male connector 40 of upper portion 16 of connector strap 18, cooperating together to provide a buckle connector 59. First and second lower straps 52, 54 are fastened together at an adjoined area 60 to keep the second connector 50 affixed to lower portion 20. First lower strap 52 connects with second lower strap 54 by fastening the plurality of lower portion patches 56 with lower portion patch 58, thereby allowing lower portion 20 to couple with knee strap 22.

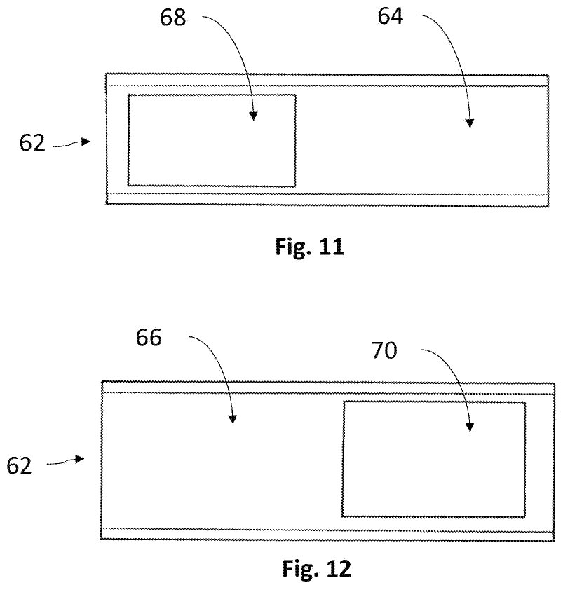

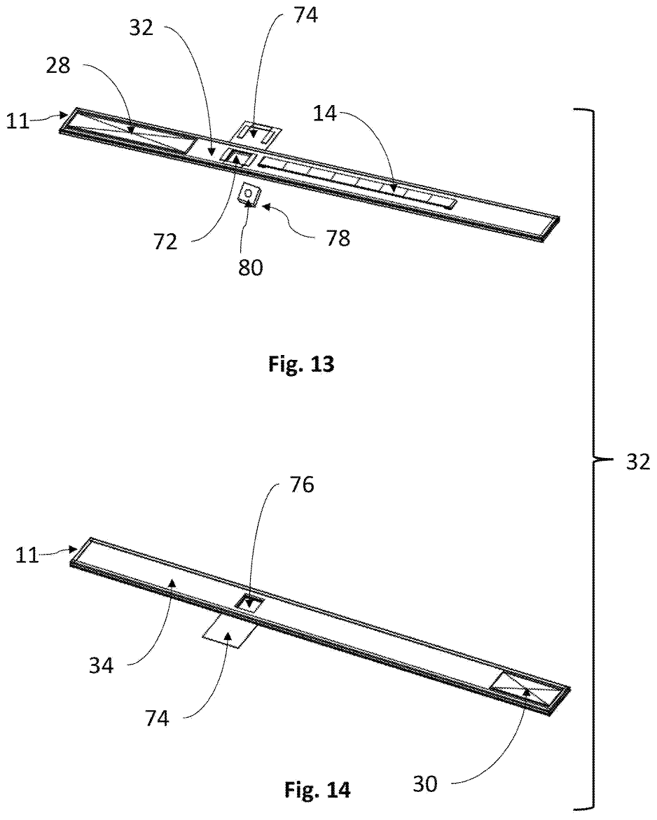

In addition to main band 11, waist band 10 may include an extension band 62 that can be fastened to main band 11 to increase the waist capacity of waist band 10. Extension band 62 includes a first extension band side 64 and a second extension band side 66, as shown in FIGS. 11 and 12. First extension band side 64 has a first extension band patch/area 68 of hook and loop fasteners, and second extension band side 66 has a second extension band patch 70 of hook and loop fasteners. In one design, first extension band patch 68 has hook fasteners, and second extension band patch 70 has loop fasteners. If waist band 10 is too short to fit around waist 8 of user 12, extension band 62 can be used to lengthen waist band 10. Instead of coupling the complementary hook and loop patches of waist band 28, 30 to each other, complementary hook and loop patches of waist band 28, 30 may be affixed to complementary hook and loop patches of the extension band 56, 58. In an alternative design, extension band 62 may be used in succession with one or more additional extension bands 62 to accommodate waist 8 of a user 12.

Knee brace mechanism 6 preferably includes an upper rigid link 23, a lower rigid link 25, and a pivot 27 that allows upper and lower rigid links 23, 25 to rotate relative to each other. Knee brace mechanism 6 further includes pads 29 positioned between upper and lower rigid links 23, 25 and a leg 24 of user 12 to conform knee brace mechanism 6 to leg 24. A plurality of auxiliary knee straps 26 are wrapped around and/or connected to knee brace mechanism 6 at different elevations on leg 24 of user 12. Knee brace stabilizer 4 is preferably positioned over clothing 31 of user 12, allowing knee brace stabilizer 4 to be removed from clothing 31 and providing universal application.

To support knee brace 6 positioned on leg 24 along with knee straps 22, waistband 28 is positioned around waist 8 of user 12 and secured by affixing complementary hook and loop fastener patches 28, 30 on waistband 28. As discussed above, extension band(s) 62 may be added to increase the length of waist band 28. Next, upper portions 16 of connector strap 18 are coupled to respective loops 14 by feeding respective first upper-portion patches 44 through loops 14 and securing first upper-portion patches 44 to second upper-portion patches 46 as shown in FIG. 1. Then, lower portions 20 of connector straps 18 are coupled to knee straps 22 by feeding first lower straps 52 of lower portions 20 behind and over respective knee straps 22 and fastening them to respective second lower straps 54. Next, male connectors 40 of upper portions 16 are coupled to respective female connectors 50. The length of connector straps 18 can then be adjusted by moving the relative positions of first-and second-upper portion patches 44, 46 relative to each other and/or moving the relative positions of first and second lower straps 52, 54.

To remove knee brace stabilizer 4, first-and second-upper portion patches 44, 46 are uncoupled to allow removal from loops 14, first and second lower straps 52, 54 are uncoupled to allow removal from knee straps 22, and patches 28, 20 are uncoupled to allow remove of waistband 28. If necessary, knee brace 6 can be removed from over the user's clothing 31 and from user 12.

An alternative embodiment main band 11 has a pocket 72 for holding a thermal sensor 78 with a thermal entry point 80, a flap 74 for covering thermal sensor 78, and a sleeve 76. Thermal sensor 78 is placed into pocket 72 such that thermal entry point 80 is facing sleeve 76. Thermal sensor 78 may be used to determine how long user 12 has been wearing knee brace stabilizer 4. For example, when thermal sensor 78 detects heat from a user's body, this detection is used to indicate that a user is wearing main band 11 and the rest of the device. When thermal sensor 78 no longer detects heat from the user's body, this is used to indicate that the user is no longer wearing the main band 11 and the rest of the device.

1. A knee brace and knee brace stabilizer assembly, comprising:
a knee brace including
a knee brace mechanism configured to restrain movement of a human knee and
a knee strap coupled to the knee brace mechanism,
a knee brace stabilizer including
a waist band configured to be supported by a human waist, the waist band including
a belt and
a plurality of loops supported by the belt, and
a connector strap that couples to at least one of the plurality of loops and couples to the knee strap to restrain movement of the knee brace relative to the waist band.

2. The knee brace and knee brace stabilizer assembly of claim 1, wherein the waist band has a length and the plurality of loops extends around less than one-half of the length of the waist band.

3. The knee brace and knee brace stabilizer assembly of claim 1, wherein the plurality of loops extend away from the belt.

4. The knee brace and knee brace stabilizer assembly of claim 1, wherein the connector strap has an end and at least one of the plurality of loops is positioned in the end of the connector strap.

5. The knee brace and knee brace stabilizer assembly of claim 1, wherein the waist band has a first side and a second side coupled to the first side by hook and loop fasteners providing the waist band with an adjustable length.

6. The knee brace and knee brace stabilizer assembly of claim 1, wherein the waist band includes a main band and an extension band configured to be coupled to the main band to increase the waist capacity of the waist band.

7. The knee brace and knee brace stabilizer assembly of claim 7, wherein the extension band has a first side that couples to a first side of the main band through hook and loop fasteners and the extension band has a second side that couples to a second side of the waist belt through hook and loop fasteners to increase the waist capacity of the waist band.

8. The knee brace and knee brace stabilizer assembly of claim 1, the knee brace and knee brace stabilizer assembly further comprising a thermal sensor, wherein the waist band includes a pocket with the thermal sensor positioned therein.

9. A knee brace and knee brace stabilizer assembly, comprising:
a knee brace including
a knee brace mechanism configured to restrain movement of a human knee and
a knee strap coupled to the knee brace mechanism,
a knee brace stabilizer including
a waist band configured to be supported by a human waist and
a connector strap that couples to the waist band to restrain movement of the knee brace relative to the waist band, the connector strap having an adjustable length to permit adjustment of the distance between the waist band and the knee strap.

10. The knee brace and knee brace stabilizer assembly of claim 9, wherein the connector strap is made from an elastic material.

11. The knee brace and knee brace stabilizer assembly of claim 9, wherein the connector strap has hook and loop fasteners on a first portion of a side of the connector strap and complementary hook and loop fasteners on a second portion of the side of the connector strap.

12. The knee brace and knee brace stabilizer assembly of claim 11, wherein the waist band includes a belt and a plurality of loops supported by the belt, a first end of the connector strap is positioned through at least one of the plurality of loops and adjustment of the first portion of the side of the connector strap relative to the second portion of the side of the connector strap provides adjustment of the adjustable length of the connector strap.

13. The knee brace and knee brace stabilizer assembly of claim 9, wherein the connect strap has a first portion, a second portion, and a buckle connecting the first portion to the second portion.

14. The knee brace and knee stabilizer assembly of claim 13, wherein the first portion has variable length and second portion has a fixed length.

15. The knee brace and knee brace stabilizer assembly of claim 9, the knee brace and knee brace stabilizer assembly further comprising a thermal sensor, wherein the waist band includes a pocket with the thermal sensor positioned therein.

16. A knee brace and knee brace stabilizer assembly, comprising:
a knee brace including
a knee brace mechanism configured to restrain movement of a human knee and
a knee strap coupled to the knee brace mechanism,
a knee brace stabilizer including
a waist band configured to be supported by a human waist and
a connector strap that couples to waist band to restrain movement of the knee brace relative to the waist band, the connector strap having an upper portion coupled to the waist band, a lower portion coupled to the knee strap, and a connector connecting the upper portion to the lower portion.

17. The knee brace and knee brace stabilizer assembly of claim 16, wherein the connector is a buckle.

18. The knee brace and knee brace stabilizer assembly of claim 16, wherein the connector has a coupled state coupling the knee brace to the waist band and uncoupled state uncoupling the knee brace from the waist band.

19. The knee brace and knee brace stabilizer assembly of claim 16, wherein the connector strap has a first portion and a second portion coupled together by the connector, the first portion has a fixed length, and the second portion has an adjustable length.

20. The knee brace and knee brace stabilizer assembly of claim 16, the knee brace and knee brace stabilizer assembly further comprising a thermal sensor, wherein the waist band includes a pocket with the thermal sensor positioned therein.
